(19) United States
(12) Patent Application Publication
(10) Pub. No.: US 2023/0401483 A1
(43) Pub. Date: Dec. 14, 2023

(54) HEARING OUTCOME PREDICTION ESTIMATOR

(71) Applicant: Advanced Bionics AG, Staefa (CH)

(72) Inventors: Josef Chalupper, Paunzhausen (DE); Gunnar Geissler, Dollbergen (DE)

(21) Appl. No.: 18/036,220

(22) PCT Filed: Nov. 20, 2020
(86) PCT No.: PCT/IB2020/060991

§ 371 Date: May 10, 2023

(51) Int. Cl. G06N 20/00; H04R 25/00

(52) U.S. Cl. CPC G06N 20/00; H04R 25/70

(57) ABSTRACT

An exemplary method includes a hearing performance prediction system aggregating a plurality of training examples, a training example in the plurality of training examples including a hearing aid dataset and a cochlear implant dataset associated with a user; and training a machine learning model using the plurality of training examples. The training may include computing, using the machine learning model, a predicted hearing performance for the user based on the hearing aid dataset of the user in the training example; computing a feedback value based on the predicted hearing performance of the user and the cochlear implant dataset of the user in the training example; and adjusting one or more model parameters of the machine learning model based on the feedback value.

### BACKGROUND INFORMATION

Although cochlear implant systems often provide significantly improved hearing capability for hearing impaired users, including those who are already using conventional hearing aids, it can sometimes be difficult for a hearing impaired user to commit to being implanted with a cochlear implant without assurance that his or her hearing will actually improve by a significant margin. However; as a number of factors (e.g., age of onset of deafness, duration of deafness, age of user when cochlear implant system is received, degree of residual hearing, user anatomy, cochlear implant system settings, etc.) can affect cochlear implant system performance for a particular user, it can currently be difficult or impossible for a hearing specialist to objectively predict potential hearing improvement that a user may obtain with a cochlear implant and thereby provide the user with this assurance.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary cochlear implant system.

FIG. 2 shows an exemplary configuration of the cochlear implant system of FIG. 1.

FIG. 3 shows another exemplary configuration of the cochlear implant system of FIG. 1.

FIG. 4 illustrates an exemplary hearing performance prediction system.

FIG. 5 shows an exemplary implementation of a hearing performance prediction system to train a machine learning model.

FIGS. 6-8 illustrate exemplary methods.

FIGS. 9-10 illustrate exemplary graphical user interfaces.

FIG. 11 illustrates an exemplary computing device.

### DETAILED DESCRIPTION

The present disclosure describes systems and methods for training and implementing a machine learning model to predict a potential hearing performance of a user with a cochlear implant system. For example, as described herein, a hearing performance prediction system may aggregate a plurality of training examples in which a training example in the plurality of training examples may include a hearing aid dataset and a cochlear implant dataset associated with a user. The hearing performance prediction system may train a machine learning model using the plurality of training examples by 1) computing, using the machine learning model, a predicted hearing performance for the user based on the hearing aid dataset of the user in the training example, 2) computing a feedback value based on the predicted hearing performance of the user and the cochlear implant dataset of the user in the training example, and 3) adjusting one or more model parameters of the machine learning model based on the feedback value.

As described herein, once a training process of the machine learning model is completed, the trained machine learning model may be implemented in an application such as a software application executed by a computing device (e.g., a fitting device in a clinical facility, an electronic device of a hearing aid user, etc.), The application may be configured to use the trained machine learning model to compute a predicted hearing performance that a hearing-impaired user may obtain if the user receives a cochlear implant system.

The systems and methods described herein are advantageous in a number of technical respects. For example, as described herein, the systems and methods may train a machine learning model with multiple training examples. Each training example may include a hearing aid dataset of a user that was generated during a first time period prior to a cochlear implant being implanted in the user, where the user was associated with (e.g., using) a hearing aid device during the first time period. The training example may also include a cochlear implant dataset of the user that was generated during a second time period subsequent to the cochlear implant being implanted in the user, where the user was associated with (e.g., using) a cochlear implant system including the cochlear implant during the second time period. Thus, the hearing aid dataset and the cochlear implant dataset may collectively indicate an impact of the cochlear implant system on the hearing performance of the user (e.g., a difference in hearing performance between when the user was using the hearing aid and when the user was using the cochlear implant system). Therefore, by training the machine learning model with hearing aid datasets and cochlear implant datasets of multiple users in multiple training examples, the trained machine learning model can be used to reliably predict a potential hearing performance that a particular user can obtain with a cochlear implant system.

Moreover, the systems and methods described herein may apply the trained machine learning model to perform such prediction for various users in an objective and consistent manner. In this manner, the subjectivity of hearing specialists in predicting the potential hearing performance of users with a cochlear implant system can be avoided.

Furthermore, in addition to the hearing aid dataset and the cochlear implant dataset, the training example may, in some embodiments, also include user data of the training example user (e.g., a user age, a user language, hearing impairment history of the user, etc.) and clinic data of a clinical facility associated with the user (e.g., a performance metric of the clinical facility, etc.). Thus, the training example may provide a comprehensive context about the hearing impairment of the training example user, historical hearing treatments applied to the training example user, and hearing performances of the training example user before and after receiving a cochlear implant system. As described herein, the systems and methods may aggregate the user data, the clinic data, the hearing aid dataset, and the cochlear implant dataset for the training example from various data sources (e.g., a mobile phone of the user, a hearing aid of the user, the clinical facility associated with the user, etc.). Moreover, the systems and methods may collect multiple training examples associated with various users that speak different languages. As a result, the systems and methods described herein may train the machine learning model with a large number of comprehensive and heterogeneous training examples. This may allow the accuracy of the machine learning model in predicting a potential hearing performance that a particular user may obtain with a cochlear implant system to be improved.

Additionally, the systems and methods described herein may generate a visual representation of a predicted hearing performance of a user with a cochlear implant system, and display the visual representation to the user on a display device. Thus, the user may reference the visual representation of the predicted hearing performance, and make an informed decision with respect to receiving a cochlear implant system. As described herein, once the user begins using the cochlear implant system, the systems and methods may also illustrate the predicted hearing performance of the user and an actual hearing performance of the user with the cochlear implant system in the same visual representation, thereby facilitating an evaluation of the hearing improvement of the user under a hearing treatment with the cochlear implant system.

As used herein, a hearing aid device (or simply hearing aid) refers to any device configured to present acoustic stimulation representative of sound to a user without also presenting electrical stimulation representative of the sound to the user. The acoustic stimulation may include, for example, an amplified version of the sound.

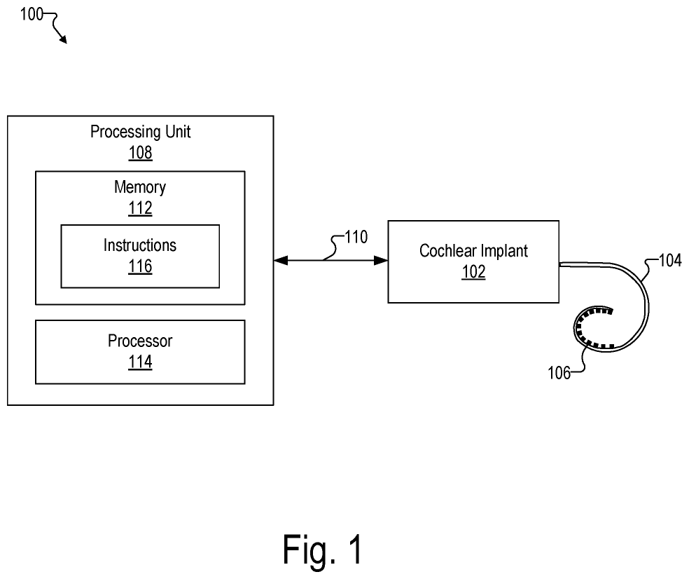

In contrast, a cochlear implant system may be configured to present electrical stimulation representative of sound to a user. For example, FIG. 1 illustrates an exemplary cochlear implant system 100 configured to be used by a recipient (also referred to herein as a user). As shown, cochlear implant system 100 includes a cochlear implant 102, an electrode lead 104 physically coupled to cochlear implant 102 and having an array of electrodes 106, and a processing unit 108 configured to be communicatively coupled to cochlear implant 102 by way of a communication link 110.

Cochlear implant system 100 shown in FIG. 1 is unilateral (i.e., associated with only one ear of the recipient). Alternatively, a bilateral configuration of cochlear implant system 100 may include separate cochlear implants and electrode leads for each ear of the recipient. In the bilateral configuration, processing unit 108 may be implemented by a single processing unit configured to interface with both cochlear implants or by two separate processing units each configured to interface with a different one of the cochlear implants.

Cochlear implant 102 may be implemented by any suitable type of implantable stimulator. For example, cochlear implant 102 may be implemented by an implantable cochlear stimulator. Additionally or alternatively, cochlear implant 102 may be implemented by a brainstem implant and/or any other type of device that may be implanted within the recipient and configured to apply electrical stimulation to one or more stimulation sites located along an auditory pathway of the recipient.

In some examples, cochlear implant 102 may be configured to generate electrical stimulation representative of an audio signal processed by processing unit 108 in accordance with one or more stimulation parameters transmitted to cochlear implant 102 by processing unit 108. Cochlear implant 102 may be further configured to apply the electrical stimulation to one or more stimulation sites (e.g., one or more intracochlear locations) within the recipient by way of one or more electrodes 106 on electrode lead 104. In some examples, cochlear implant 102 may include a plurality of independent current sources each associated with a channel defined by one or more of electrodes 106. In this manner, different stimulation current levels may be applied to multiple stimulation sites simultaneously by way of multiple electrodes 106.

Cochlear implant 102 may additionally or alternatively be configured to generate, store, and/or transmit data. For example, cochlear implant may use one or more electrodes 106 to record one or more signals (e.g., one or more voltages, impedances, evoked responses within the recipient, and/or other measurements) and transmit, by way of communication link 110, data representative of the one or more signals to processing unit 108. In some examples, this data is referred to as back telemetry data.

Electrode lead 104 may be implemented in any suitable manner. For example, a distal portion of electrode lead 104 may be pre-curved such that electrode lead 104 conforms with the helical shape of the cochlea after being implanted, Electrode lead 104 may alternatively be naturally straight or of any other suitable configuration.

In some examples, electrode lead 104 includes a plurality of wires (e.g., within an outer sheath) that conductively couple electrodes 106 to one or more current sources within cochlear implant 102. For example, if there are n electrodes 106 on electrode lead 104 and n current sources within cochlear implant 102, there may be n separate wires within electrode lead 104 that are configured to conductively connect each electrode 106 to a different one of the n current sources. Exemplary values for n are 8, 12, 16, or any other suitable number.

Electrodes 106 are located on at least a distal portion of electrode lead 104. In this configuration, after the distal portion of electrode lead 104 is inserted into the cochlea, electrical stimulation may be applied by way of one or more of electrodes 106 to one or more intracochlear locations. One or more other electrodes (e.g., including a ground electrode, not explicitly shown) may also be disposed on other parts of electrode lead 104 (e.g., on a proximal portion of electrode lead 104) to, for example, provide a current return path for stimulation current applied by electrodes 106 and to remain external to the cochlea after the distal portion of electrode lead 104 is inserted into the cochlea. Additionally or alternatively, a housing of cochlear implant 102 may serve as a ground electrode for stimulation current applied by electrodes 106.

Processing unit 108 may be configured to interface with (e.g., control and/or receive data from) cochlear implant 102. For example, processing unit 108 may transmit commands (e.g., stimulation parameters and/or other types of operating parameters in the form of data words included in a forward telemetry sequence) to cochlear implant 102 by way of communication link 110, Processing unit 108 may additionally or alternatively provide operating power to cochlear implant 102 by transmitting one or more power signals to cochlear implant 102 by way of communication link 110. Processing unit 108 may additionally or alternatively receive data from cochlear implant 102 by way of communication link 110. Communication link 110 may be implemented by any suitable number of wired and/or wireless bidirectional and/or unidirectional links.

As shown, processing unit 108 includes a memory 112 and a processor 114 configured to be selectively and communicatively coupled to one another. In some examples, memory 112 and processor 114 may be distributed between multiple devices and/or multiple locations as may serve a particular implementation.

Memory 112 may be implemented by any suitable non-transitory computer-readable medium and/or non-transitory processor-readable medium, such as any combination of non-volatile storage media and/or volatile storage media. Exemplary non-volatile storage media include, but are not limited to, read-only memory, flash memory, a solid-state drive, a magnetic storage device (e.g., a hard drive), ferroelectric random-access memory (“RAM”), and an optical disc, Exemplary volatile storage media include; but are not limited to, RAM (e.g., dynamic RAM).

Memory 112 may maintain (e.g., store) executable data used by processor 114 to perform one or more of the operations described herein. For example, memory 112 may store instructions 116 that may be executed by processor 114 to perform any of the operations described herein. Instructions 116 may be implemented by any suitable application, program (e.g., sound processing program), software, code, and/or other executable data instance. Memory 112 may also maintain any data received, generated, managed, used, and/or transmitted by processor 114.

Processor 114 may be configured to perform (e.g., execute instructions 116 stored in memory 112 to perform) various operations with respect to cochlear implant 102.

To illustrate, processor 114 may be configured to control an operation of cochlear implant 102. For example, processor 114 may receive an audio signal (e.g., by way of a microphone communicatively coupled to processing unit 108, a wireless interface (e.g., a Bluetooth interface), and/or a wired interface (e.g., an auxiliary input port)). Processor 114 may process the audio signal in accordance with a sound processing program (e.g., a sound processing program stored in memory 112) to generate appropriate stimulation parameters. Processor 114 may then transmit the stimulation parameters to cochlear implant 102 to direct cochlear implant 102 to apply electrical stimulation representative of the audio signal to the recipient.

In some implementations, processor 114 may also be configured to apply acoustic stimulation to the recipient. For example, a receiver (also referred to as a loudspeaker) may be optionally coupled to processing unit 108. In this configuration, processor 114 may deliver acoustic stimulation to the recipient by way of the receiver. The acoustic stimulation may be representative of an audio signal (e.g., an amplified version of the audio signal), configured to elicit an evoked response within the recipient, and/or otherwise configured. In configurations in which processor 114 is configured to both deliver acoustic stimulation to the recipient and direct cochlear implant 102 to apply electrical stimulation to the recipient, cochlear implant system 100 may be referred to as a bimodal hearing system and/or any other suitable term.

Processor 114 may be additionally or alternatively configured to receive and process data generated by cochlear implant 102. For example, processor 114 may receive data representative of a signal recorded by cochlear implant 102 using one or more electrodes 106 and, based on the data, adjust one or more operating parameters of processing unit 108. Additionally or alternatively, processor 114 may use the data to perform one or more diagnostic operations with respect to cochlear implant 102 and/or the recipient.

Other operations may be performed by processor 114 as may serve a particular implementation. In the description provided herein, any references to operations performed by processing unit 108 and/or any implementation thereof may be understood to be performed by processor 114 based on instructions 116 stored in memory 112.

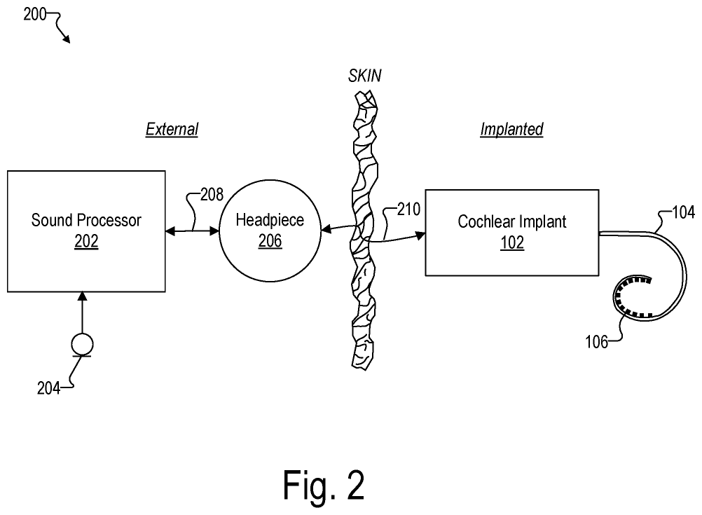

Processing unit 108 may be implemented by one or more devices configured to interface with cochlear implant 102. To illustrate, FIG. 2 shows an exemplary configuration 200 of cochlear implant system 100 in which processing unit 108 is implemented by a sound processor 202 configured to be located external to the recipient. In configuration 200, sound processor 202 is communicatively coupled to a microphone 204 and to a headpiece 206 that are both configured to be located external to the recipient.

Sound processor 202 may be implemented by any suitable device that may be worn or carried by the recipient. For example, sound processor 202 may be implemented by a behind-the-ear (“BTE”) unit configured to be worn behind and/or on top of an ear of the recipient. Additionally or alternatively, sound processor 202 may be implemented by an off-the-ear unit (also referred to as a body worn device) configured to be worn or carried by the recipient away from the ear, Additionally or alternatively, at least a portion of sound processor 202 is implemented by circuitry within headpiece 206.

Microphone 204 is configured to detect one or more audio signals (e.g., that include speech and/or any other type of sound) in an environment of the recipient. Microphone 204 may be implemented in any suitable manner. For example, microphone 204 may be implemented by a microphone that is configured to be placed within the concha of the ear near the entrance to the ear canal, such as a T-MIC™ microphone from Advanced Bionics. Such a microphone may be held within the concha of the ear near the entrance of the ear canal during normal operation by a boom or stalk that is attached to an ear hook configured to be selectively attached to sound processor 202. Additionally or alternatively, microphone 204 may be implemented by one or more microphones in or on headpiece 206, one or more microphones in or on a housing of sound processor 202, one or more beam-forming microphones, and/or any other suitable microphone as may serve a particular implementation.

Headpiece 206 may be selectively and communicatively coupled to sound processor 202 by way of a communication link 208 (e.g., a cable or any other suitable wired or wireless communication link), which may be implemented in any suitable manner. Headpiece 206 may include an external antenna (e.g., a coil and/or one or more wireless communication components) configured to facilitate selective wireless coupling of sound processor 202 to cochlear implant 102, Headpiece 206 may additionally or alternatively be used to selectively and wirelessly couple any other external device to cochlear implant 102. To this end, headpiece 206 may be configured to be affixed to the recipient's head and positioned such that the external antenna housed within headpiece 206 is communicatively coupled to a corresponding implantable antenna (which may also be implemented by a coil and/or one or more wireless communication components) included within or otherwise connected to cochlear implant 102. In this manner, stimulation parameters and/or power signals may be wirelessly and transcutaneously transmitted between sound processor 202 and cochlear implant 102 by way of a wireless communication link 210.

In configuration 200, sound processor 202 may receive an audio signal detected by microphone 204 by receiving a signal (e.g., an electrical signal) representative of the audio signal from microphone 204. Sound processor 202 may additionally or alternatively receive the audio signal by way of any other suitable interface as described herein. Sound processor 202 may process the audio signal in any of the ways described herein and transmit, by way of headpiece 206, stimulation parameters to cochlear implant 102 to direct cochlear implant 102 to apply electrical stimulation representative of the audio signal to the recipient.

In an alternative configuration, sound processor 202 may be implanted within the recipient instead of being located external to the recipient. In this alternative configuration, which may be referred to as a fully implantable configuration of cochlear implant system 100, sound processor 202 and cochlear implant 102 may be combined into a single device or implemented as separate devices configured to communicate one with another by way of a wired and/or wireless communication link. In a fully implantable implementation of cochlear implant system 100, headpiece 206 may not be included and microphone 204 may be implemented by one or more microphones implanted within the recipient, located within an ear canal of the recipient, and/or external to the recipient.

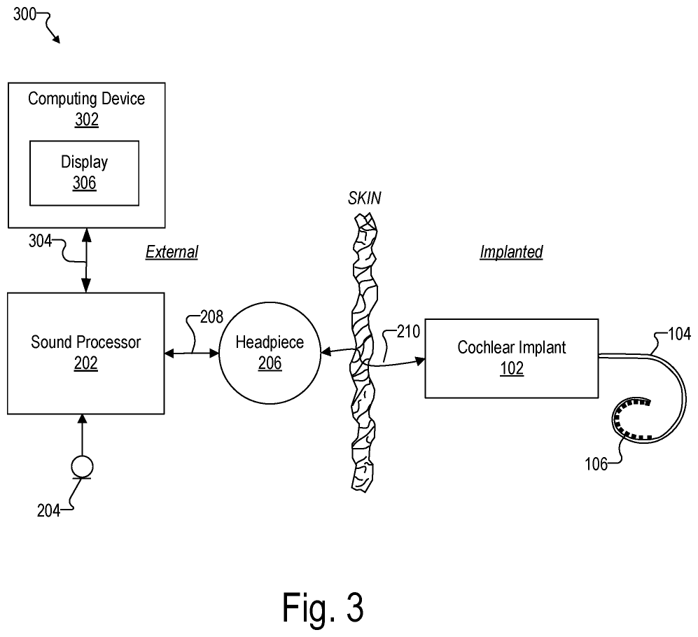

FIG. 3 shows an exemplary configuration 300 of cochlear implant system 100 in which processing unit 108 is implemented by a combination of sound processor 202 and a computing device 302 configured to communicatively couple to sound processor 202 by way of a communication link 304, which may be implemented by any suitable wired or wireless communication link.

Computing device 302 may be implemented by any suitable combination of hardware and software. To illustrate, computing device 302 may be implemented by a mobile device (e.g., a mobile phone, a laptop, a tablet computer, etc.), a desktop computer, and/or any other suitable computing device as may serve a particular implementation. As an example, computing device 302 may be implemented by a mobile device configured to execute an application (e.g., a “mobile app”) that may be used by a user (e.g., the recipient, a clinician, and/or any other user) to control one or more settings of sound processor 202 and/or cochlear implant 102 and/or perform one or more operations (e.g., diagnostic operations) with respect to data generated by sound processor 202 and/or cochlear implant 102.

In some examples, computing device 302 may be configured to control an operation of cochlear implant 102 by transmitting one or more commands to cochlear implant 102 by way of sound processor 202. Likewise, computing device 302 may be configured to receive data generated by cochlear implant 102 by way of sound processor 202. Alternatively, computing device 302 may interface with (e.g., control and/or receive data from) cochlear implant 102 directly by way of a wireless communication link between computing device 302 and cochlear implant 102. In some implementations in which computing device 302 interfaces directly with cochlear implant 102, sound processor 202 may or may not be included in cochlear implant system 100.

Computing device 302 is shown as having an integrated display 306. Display 306 may be implemented by a display screen, for example, and may be configured to display content generated by computing device 302. Additionally or alternatively, computing device 302 may be communicatively coupled to an external display device (not shown) configured to display the content generated by computing device 302.

In some examples, computing device 302 represents a fitting device configured to be selectively used (e.g., by a clinician) to fit sound processor 202 and/or cochlear implant 102 to the recipient. In these examples, computing device 302 may be configured to execute a fitting program configured to set one or more operating parameters of sound processor 202 and/or cochlear implant 102 to values that are optimized for the recipient. As such, in these examples, computing device 302 may not be considered to be part of cochlear implant system 100. Instead, computing device 302 may be considered to be separate from cochlear implant system 100 such that computing device 302 may be selectively coupled to cochlear implant system 100 when it is desired to fit sound processor 202 and/or cochlear implant 102 to the recipient.

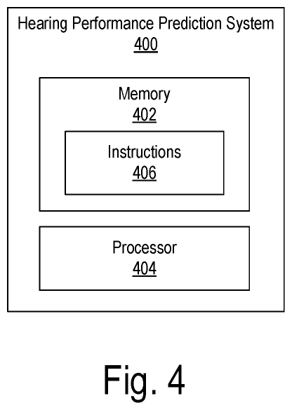

FIG. 4 shows an exemplary hearing performance prediction system 400 (“system 400”). In some embodiments, system 400 may be implemented by one or more computing devices that are not included in a cochlear implant system. For example, system 400 may be implemented by one or more servers located remote from cochlear implant system 100 described herein. In some embodiments, system 400 may be operated by and/or otherwise associated with a manufacturer of cochlear implant systems, a provider of cochlear implant systems, and/or other entities as may serve a particular implementation.

As depicted in FIG. 4, system 400 may include a memory 402 and a processor 404 configured to be selectively and communicatively coupled to one another. In some embodiments, memory 402 and processor 404 may be distributed between multiple devices and/or multiple locations as may serve a particular implementation.

In some embodiments, memory 402 may be implemented by any suitable non-transitory computer-readable medium and/or non-transitory processor-readable medium, such as any combination of non-volatile storage media and/or volatile storage media as described herein. In some embodiments, memory 402 may maintain (e.g., store) executable data used by processor 404 to perform one or more operations of system 400 described herein. For example, memory 402 may store instructions 406 executable by processor 404 to train and/or implement a machine learning model to predict a hearing performance that a user may obtain with a cochlear implant system. It should be understood that instructions 406 may be implemented by any suitable application, program, software, code, and/or other executable data instance. In some embodiments, memory 402 may also maintain any data generated, managed, used, transmitted, and/or received by processor 404.

In some embodiments, processor 404 may be configured to perform various operations of system 400 described herein. For example, processor 404 may perform one or more operations based on instructions 406 stored in memory 402 to train and/or implement a machine learning model to predict a hearing performance with a cochlear implant system for a user as described herein.

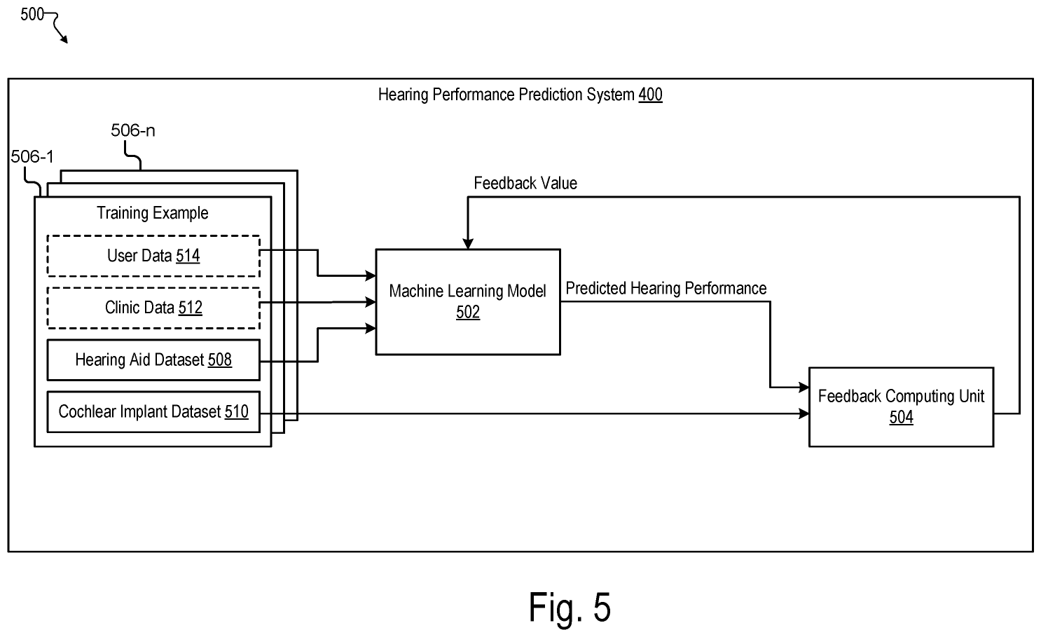

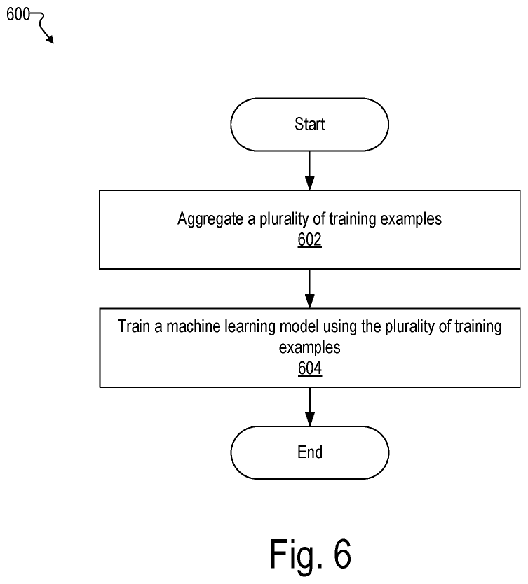

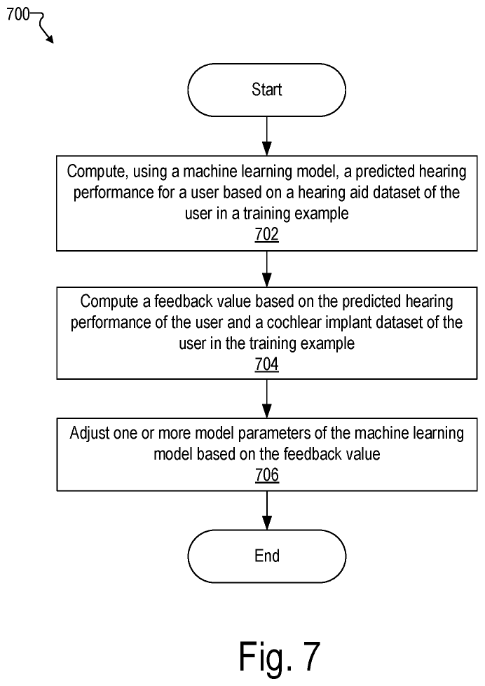

FIG. 5 illustrates a block diagram 500 of an example training stage in which system 400 may train a machine learning model. As depicted in FIG. 5, system 400 may include a machine learning model 502 and a feedback computing unit 504 that can be used to train machine learning model 502. In some embodiments, machine learning model 502 may be trained with a plurality of training examples 506 (e.g., training example 506-1 through 506-N) to compute a predicted hearing performance that a user may obtain with a cochlear implant system. As described herein, each training example 506 may include a hearing aid dataset 508 and a cochlear implant dataset 510. Each training example 506 may also optionally include clinic data 512 and/or user data 514, The training stage of machine learning model 502 is described in detail herein with reference to FIGS. 6 and 7.

FIG. 6 illustrates an exemplary method 600 that may be performed by system 400. While FIG. 6 illustrates exemplary operations according to one embodiment, other embodiments may omit, add to, reorder, and/or modify one or more operations of the method 600 depicted in FIG. 6. Each operation of the method 600 depicted in FIG. 6 may be performed in any manner described herein.

At operation 602, system 400 may aggregate a plurality of training examples (e.g., training examples 506) that can be used to train machine learning model 502. In some embodiments, each training example in the plurality of training examples may include a hearing aid dataset (e.g., hearing aid dataset 508) and a cochlear implant dataset (e.g., cochlear implant dataset 510) associated with a particular user.

A user associated with a training example may be a hearing-impaired person that is associated with (e.g., uses) a hearing aid during a first time period and then is associated with (e.g., uses) a cochlear implant system during a second time period subsequent to the first time period. In the examples provided herein, references to a user using a cochlear implant system or simply a cochlear implant are used interchangeably to refer to the user using a cochlear implant system.

As an example, the user may be in a first user state during a first time period prior to an implant surgical operation in which a cochlear implant is implanted within the user. While in the first user state during the first time period, the user may be associated with a hearing aid device and may frequently or occasionally use the hearing aid device to address the hearing impairment of the user. Subsequent to the implant surgical operation, the user may be in a second user state during a second time period. While in the second state during the second time period, the user may be associated with the cochlear implant of a cochlear implant system (e.g., system 100) and may use the cochlear implant system instead of the hearing aid device to address the hearing impairment of the user.

In some embodiments, to aggregate the training example associated with the user, system 400 may collect the hearing aid dataset of the user that is generated during the first time period in which the user is associated with the hearing aid device.

The hearing aid dataset may include various types of data associated the hearing aid device and/or with the user while the user is associated with the hearing aid device. For example, in some embodiments, the hearing aid dataset may include data representative of one or more fitting parameters of the hearing aid device, a usage pattern of the user in using the hearing aid device during the first time period, one or more hearing performance results of the user with the hearing aid device during the first time period, one or more hearing performance results of the user without the hearing aid device during the first time period, etc. Other types of data in the hearing aid dataset are also possible and contemplated.

As used herein, fitting parameters of the hearing aid device may include data representative of one or more sound processing programs used by the hearing aid device, one or more values of one or more device parameters of the hearing aid device that are adjusted to specifically adapt operations of the hearing aid device to the user, etc.

As used herein, the usage pattern of the user in using the hearing aid device may indicate a pattern in which the user uses the hearing aid device. For example, such usage pattern may indicate an average amount of time that the user uses the hearing aid device per day, a time of day and a day of week during which the user usually uses the hearing aid device, a time of day and a day of week during which the user usually does not use the hearing aid device, etc.

As used herein, the hearing performance result of the user may include one or more test results of one or more hearing performance tests and/or other medical exams that reflect a hearing performance of the user. For example, the hearing performance result may include a speech score indicating a ratio between a number of words correctly identified by the user and a number of words presented to the user. In some embodiments, the hearing performance result may additionally or alternatively include a speech audiometry of the user indicating a set of speech detection thresholds and/or a set of speech recognition thresholds corresponding to a set of predetermined frequencies (e.g., 250 Hz, 500 Hz, 1000 Hz, etc.). A speech detection threshold may indicate a lowest volume at which the user can hear a word presented to the user at a given frequency (e.g., 60 dB HL at 250 Hz). A speech recognition threshold may indicate a lowest volume at which the user can identify and repeat a word presented to the user at a given frequency (e.g., 70 dB HL at 250 Hz). In some embodiments, the hearing performance result may also include a tone detection result of the user, neural response imaging (NRI) data of the user, etc. Other types of hearing performance results are also possible and contemplated.

As described herein, the hearing aid dataset may include one or more hearing performance results of the user with the hearing aid device during the first time period. These hearing performance results may be obtained at one or more timestamps during the first time period with the user wearing the hearing aid device. As described herein, the hearing aid dataset may also include one or more hearing performance results of the user without the hearing aid device during the first time period. These hearing performance results may be obtained at one or more timestamps during the first time period with the user not wearing the hearing aid device.

In some embodiments, system 400 may collect the hearing aid dataset of the user from various data sources. For example, to collect the hearing aid dataset of the user, system 400 may receive the fitting parameters of the hearing aid device of the user from a clinical facility associated with the user. The clinical facility may communicatively couple to system 400 via a network. In some embodiments, a hearing specialist (e.g., an audiologist, an otolaryngologist, etc.) of the clinical facility may perform fitting operations to adjust the hearing aid device based on the user, and the clinical facility may then transmit the fitting parameters of the hearing aid device to system 400 via the network.

In some embodiments, to collect the hearing aid dataset of the user, system 400 may receive the hearing performance results of the user from the clinical facility associated with the user and/or from one or more electronic devices of the user. For example, similar to the fitting parameters of the hearing aid device, the hearing specialist of the clinical facility may administer hearing performance tests and/or other related medical exams with and without the hearing aid device to the user at various timestamps during the first time period. The clinical facility may then transmit hearing performance results of the user in these hearing performance tests and/or medical exams to system 400 via the network. Additionally or alternatively, the user may install a software application to perform hearing performance tests on his or her electronic device (e.g., a mobile phone, a tablet, a laptop, etc.). The electronic device of the user may communicatively couple to system 400 via the network. In some embodiments, the user may conduct hearing performance tests with and without the hearing aid device at various timestamps during the first time period using the software application on the electronic device. The electronic device may then transmit hearing performance results of the user in these hearing performance tests to system 400 via the network.

In some embodiments, to collect the hearing aid dataset of the user, system 400 may receive usage data associated with the hearing aid device of the user from the hearing aid device. The usage data may describe operations of the hearing aid device as the hearing aid device is used by the user (e.g., active time, inactive time, device parameters, etc.), In some embodiments, system 400 may determine the usage pattern of the user in using the hearing aid device based on the usage data of the hearing aid device. For example, system 400 may analyze the usage data of the hearing aid device, and determine the average amount of time that the user uses the hearing aid device per day, a time window during which the user usually uses the hearing aid device, a time window during which the user usually does not use the hearing aid device, etc.

In some embodiments, in addition to collecting the hearing aid dataset of the user that is generated during the first time period, system 400 may collect the cochlear implant dataset of the user that is generated during the second time period in which the user is associated with a cochlear implant of a cochlear implant system. The cochlear implant dataset may include various types of data associated the cochlear implant system and/or with the user while the user is associated with the cochlear implant system. For example, the cochlear implant dataset may include one or more of fitting parameters of the cochlear implant, a usage pattern of the user in using the cochlear implant, one or more hearing performance results of the user with the cochlear implant during the second time period, one or more hearing performance results of the user without the cochlear implant during the second time period, etc. Other types of data in the cochlear implant dataset are also possible and contemplated.

As used herein, the fitting parameters of the cochlear implant may include one or more values of one or more parameters that specify operations of one or more components of the cochlear implant system. For example, the fitting parameters may be representative of one or more sound processing programs executed by a processing unit of the cochlear implant system, one or more fitting parameters of the cochlear implant system that are adjusted based on the user to specifically adapt operations of the cochlear implant system to the user, etc.

As used herein, the usage pattern of the user in using the cochlear implant may indicate a pattern in which the user uses the cochlear implant system. For example, such usage pattern may indicate an average amount of time that the user uses the cochlear implant system per day, a time of day and a day of week during which the user usually uses the cochlear implant system, a time of day and a day of week during which the user usually does not use the cochlear implant system, etc.

As described herein, the cochlear implant dataset may include the hearing performance results of the user with the cochlear implant during the second time period. These hearing performance results may be obtained at one or more timestamps during the second time period with the user using the cochlear implant system. As described herein, the cochlear implant dataset may also include one or more hearing performance results of the user without the cochlear implant during the second time period. These hearing performance results may be obtained at one or more timestamps during the second time period with the user not using the cochlear implant system.

In some embodiments, system 400 may collect the cochlear implant dataset of the user from various data sources. For example, similar to the hearing aid dataset, system 400 may receive the fitting parameters of the cochlear implant of the user from a clinical facility. System 400 may also receive the hearing performance results of the user with and without the cochlear implant during the second time period from the clinical facility, an electronic device of the user, and/or any other source as may serve a particular implementation. System 400 may also receive usage data associated with the cochlear implant from one or more components of the cochlear implant system, and determine a usage pattern of the user in using the cochlear implant based on the usage data of the cochlear implant.

In some embodiments, in addition to the hearing aid dataset and the cochlear implant dataset, the training example associated with the user may also include user data (e.g., user data 514) of the user. In some embodiments, the user data may include data representative of an age of the user, etiological data of the user, pre-surgery data of the user. The user data may also include data representative of electroencephalogram (EEG) test result, heart rate, type of user activity (moving, running, sitting, turning, etc.), user location (restaurant, school, etc.), and/or any other attribute of the user.

As used herein, etiological data may describe the hearing impairment of the user and may include data representative of one or more of a cause of the hearing impairment (e.g., inner ear damage, ruptured eardrum, etc.), a hearing impairment start point indicating a time at which the hearing impairment of the user started, a hearing impairment duration indicating a time duration during which the user has experienced the hearing impairment, etc.

As used herein, the pre-surgery (pre-operative) data of the user may include one or more test results of one or more tests (e.g., auditory system tests and/or cognitive tests) performed on the user prior to the cochlear implant being implanted within the user. Non-limiting examples of such tests include, but are not limited to, a cognitive test, a working memory test, a tinnitus test, a vestibularis function test, etc. In some embodiments, the user data of the training example may also include data representative of a language of the user.

In some embodiments, the training example associated with the user may further include clinic data (e.g., clinic data 512) of the clinical facility associated with the user. The clinic data may include one or more performance metrics of the clinical facility such as a feedback metric from patients of the clinical facility, a ranking level of the clinical facility, an average performance metric of physicians associated with the clinical facility, etc.

In some embodiments, system 400 may aggregate the plurality of training examples associated with various users that are located in different geographical areas (e.g., Europe, Asia, Africa, etc.) and/or speak different languages (e.g., English, German, Mandarin, etc.). Thus, system 400 may collect a large number of heterogenous training examples to train machine learning model 502. In some embodiments, the training examples may be subjected to one or more preprocessing operations (e.g., converting measurement units, data normalization, etc.) so that these training examples may conform to the same format and structure before being used to train machine learning model 502.

At operation 604, system 400 may train machine learning model 502 using the plurality of training examples. Once trained, machine learning model 502 may be used to compute a predicted hearing performance that a user may obtain with a cochlear implant system.

In some embodiments, machine learning model 502 may be implemented using one or more supervised and/or unsupervised learning algorithms. For example, machine learning model 502 may be implemented in the form of a linear regression model, a logistic regression model, a Support Vector Machine (SVM) model, and/or other learning models. Additionally or alternatively, machine learning model 502 may be implemented in the form of a neural network including an input layer, one or more hidden layers, and an output layer. Non-limiting examples of the neural network include, but are not limited to, Convolutional Neural Network (CNN), Recurrent Neural Network (RNN), Long Short-Term Memory (LSTM) neural network, etc. Other system architectures for implementing machine learning model 502 are also possible and contemplated.

An exemplary training stage of machine learning model 502 is illustrated in FIG. 5. As depicted in FIG. 5, system 400 may input the plurality of training examples into machine learning model 502, and train machine learning model 502 with a training example from the plurality of training examples in each training cycle of the training stage.

An exemplary method 700 for training machine learning model 502 with a training example associated with a user is depicted in FIG. 7. While FIG. 7 illustrates exemplary operations according to one embodiment, other embodiments may omit, add to, reorder, and/or modify one or more operations of the method 700 depicted in FIG. 7. Each operation of the method 700 depicted in FIG. 7 may be performed in any manner described herein.

At operation 702, system 400 may compute, using machine learning model 502, a predicted hearing performance for the user based on a hearing aid dataset (e.g., hearing aid dataset 508) of the user in the training example.

In some embodiments, to compute the predicted hearing performance for the user, system 400 may apply machine learning model 502 to the hearing aid dataset of the user, and machine learning model 502 may compute a predicted hearing performance with a cochlear implant for the user based on the hearing aid dataset. In some embodiments, the predicted hearing performance may include one or more predicted hearing performance results that machine learning model 502 estimates the user may obtain with the cochlear implant at one or more timestamps during a second time period. As described herein, the second time period may be subsequent to an implant surgical operation during which the cochlear implant is implanted within the user. Accordingly, machine learning model 502 may predict the hearing performance of the user with the cochlear implant during the second time period from the hearing aid dataset generated when the user is associated with the hearing aid device during the first time period.

In some embodiments, in addition to the hearing aid dataset of the user, the computing of the predicted hearing performance of the user with the cochlear implant may be further based on user data (e.g., user data 514) of the user and/or clinic data (e.g., clinic data 512) of a clinical facility associated with the user. For example, as depicted in FIG. 5, system 400 may apply machine learning model 502 to the hearing aid dataset of the user, the user data of the user, and the clinic data of the clinical facility. In this example, machine learning model 502 may compute the predicted hearing performance with the cochlear implant for the user based on this combined set of data.

At operation 704, system 400 may compute a feedback value based on the predicted hearing performance of the user and the cochlear implant dataset of the user in the training example. For example, as depicted in FIG. 5, system 400 may input the predicted hearing performance of the user with the cochlear implant computed by machine learning model 502 and the cochlear implant dataset of the user in the training example into the feedback computing unit 504. As described herein, the predicted hearing performance of the user may include one or more predicted hearing performance results which machine learning model 502 estimates the user may obtain with the cochlear implant at one or more timestamps during the second time period after the implant surgical operation. In contrast, the cochlear implant dataset of the user in the training example may include one or more hearing performance results that the user actually obtained with the cochlear implant at these one or more timestamps during the second time period.

In some embodiments, the feedback computing unit 504 may compute the feedback value based on the predicted hearing performance of the user and the cochlear implant dataset of the user. For example, the feedback value may be a mean squared error between the predicted hearing performance results of the user with the cochlear implant computed by machine learning model 502 and the corresponding hearing performance results of the user with the cochlear implant in the cochlear implant dataset of the training example. Other implementations for computing the feedback value are also possible and contemplated.

At operation 706, system 400 may adjust one or more model parameters of machine learning model 502 based on the feedback value. For example, as depicted in FIG. 5, system 400 may back-propagate the feedback value computed by the feedback computing unit 504 to machine learning model 502, and adjust the model parameters of machine learning model 502 (e.g., weight values assigned to various data elements in the training examples) based on the feedback value.

In some embodiments, system 400 may determine whether the model parameters of machine learning model 502 have been sufficiently adjusted. For example, system 400 may determine that machine learning model 502 has been subjected to a predetermined number of training cycles and therefore has been trained with a predetermined number of training examples. Additionally or alternatively, system 400 may determine that the feedback value satisfies a predetermined feedback value threshold, and thus determine that the model parameters of machine learning model 502 have been sufficiently adjusted. Additionally or alternatively, system 400 may determine that the feedback value remains substantially unchanged for a predetermined number of training cycles (e.g., a difference between the feedback values computed in sequential training cycles satisfying a difference threshold), and thus determine that the model parameters of machine learning model 502 have been sufficiently adjusted.

In some embodiments, responsive to determining that the model parameters of machine learning model 502 have been sufficiently adjusted, system 400 may determine that the training stage of machine learning model 502 is completed, and select the current values of the model parameters to be values of the model parameters in trained machine learning model 502. It should be understood that system 400 may continue to aggregate the training examples and train machine learning model 502 with the aggregated training examples over time. For example, when system 400 collects one or more additional training examples from one or more data sources, system 400 may update the plurality of training examples to include both existing training examples and the additional training examples, and train machine learning model 502 with the updated plurality of training examples according to the training process described herein. Additionally or alternatively, system 400 may periodically collect additional training examples from one or more data sources, update the plurality of training examples to include both existing training examples and the additional training examples, and train machine learning model 502 with the updated plurality of training examples at predetermined intervals.

In some embodiments, system 400 may implement trained machine learning model 502 in an application associated with a hearing aid device to use that machine learning model 502 in an implementation stage. In some embodiments, the application may be a software application executed by a computing device associated with one or more hearing aid devices of one or more users. The application may be configured to perform one or more hearing performance tests for the users and/or perform one or more fitting operations for the hearing aid devices of the users.

As an example, system 400 may implement trained machine learning model 502 in an application installed on an electronic device (e.g., a mobile phone) of a user who is under a hearing treatment with a hearing aid device. The electronic device of the user may be communicatively coupled to the hearing aid device of the user, and the user may use the application on the electronic device to monitor and/or control operations of the hearing aid device. The user may also use the application to perform one or more hearing performance tests with and without the hearing aid device. As another example, system 400 may implement trained machine learning model 502 in an application installed on a clinical device of a clinic facility. The clinical device may be used to administer hearing performance tests for one or more users who are under a hearing treatment with a hearing aid device in the clinic facility or to perform fitting operations for one or more hearing aid devices of the users.

In some embodiments, for a user currently in a first state in which the user is associated with a hearing aid device, the user and/or a hearing specialist of the user may use the application to perform a hearing performance test with the hearing aid device and/or a hearing performance test without the hearing aid device for the user, and the application may analyze hearing performance results of the user in these hearing performance tests. Alternatively, the application may receive the hearing performance results of the user in these hearing performance tests from one or more data sources (e.g., a computing device of the clinic facility, an electronic device of the user, the hearing aid device of the user, etc.), and analyze the hearing performance results of the user in these hearing performance tests. In some embodiments, to analyze a hearing performance result of the user in a hearing performance test, the application may determine whether the hearing performance result of the user in the hearing performance test satisfies one or more thresholds (e.g., a first hearing performance threshold and a second hearing performance threshold).

In some embodiments, if the hearing performance result of the user in the hearing performance test satisfies the first hearing performance threshold and does not satisfy the second hearing performance threshold (e.g., a speech score of the user is lower than a first predetermined score such as 50% but not lower than a second predetermined score such as 20%), the application may determine that the hearing aid device appears to improve a hearing performance of the user, and that such improvement may be increased by adjusting one or more settings of the hearing aid device or switching to a different hearing aid device. In this case, the application may automatically display (e.g., by way of any suitable display device) a recommendation to adjust fitting parameters of the hearing aid device so that operations of the hearing aid device are optimally adapted to the user. Additionally or alternatively, the application may automatically display a recommendation to upgrade the hearing aid device of the user to a different hearing aid device (e.g., a hearing aid device that can provide a higher gain and/or a higher maximum power output to the user).

In some embodiments, if the hearing performance result of the user in the hearing performance test satisfies both the first hearing performance threshold and the second hearing performance threshold (e.g., the speech score of the user is lower than both the first predetermined score such as 50% and the second predetermined score such as 20%), the application may determine that the hearing aid device appears to only marginally improve a hearing performance of the user and that the user may be better served by a cochlear implant system. In this case, the application may use trained machine learning model 502 to predict a hearing performance with a cochlear implant system for the user, thereby facilitating the user and the hearing specialist of the user in determining whether a hearing treatment with a cochlear implant system instead of the hearing aid device is potentially effective for the user.

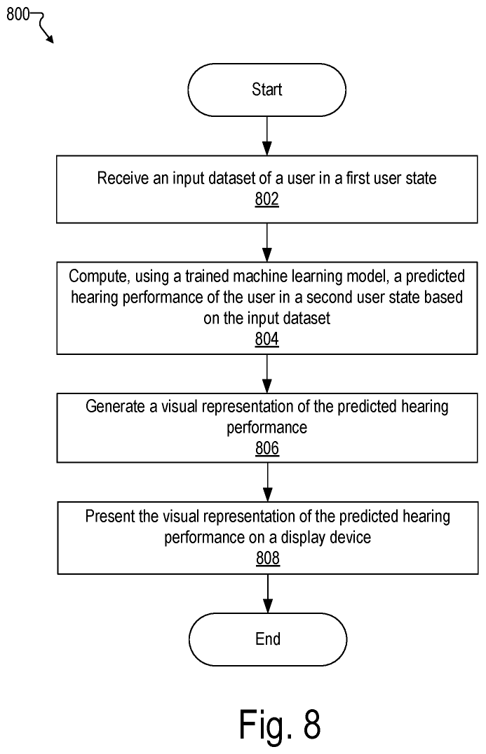

An exemplary method 800 for applying machine learning model 502 in the implementation stage to predict a hearing performance of a user is depicted in FIG. 8. The method 800 may be performed by an application executed by a computing device to compute a predicted hearing performance with a cochlear implant for a user who is currently associated with a hearing aid device. While FIG. 8 illustrates exemplary operations according to one embodiment, other embodiments may omit, add to, reorder, and/or modify one or more operations of the method 800 depicted in FIG. 8. Each operation of the method 800 depicted in FIG. 8 may be performed in any manner described herein.

At operation 802, the application may receive an input dataset of a user in a first user state. In some embodiments, when the user is in the first user state, the user is currently associated with a hearing aid device, and thus the user frequently or occasionally uses the hearing aid device to treat his or her hearing impairment. The input dataset may include a hearing aid dataset, user data, and/or clinic data associated with the user in the first user state. Each of these types of data may be as described herein. Other types of data in the input dataset are also possible and contemplated. In some embodiments, the hearing specialist and/or the user may enter the input dataset directly into the application via a graphical user interface. Additionally or alternatively, the application may receive the input dataset in any other way described herein.

At operation 804, the application may use trained machine learning model 502 to compute a predicted hearing performance of the user in a second user state based on the input dataset. In some embodiments, to change from the current first user state to the second user state, the user will be subjected to an implant surgical operation to implant a cochlear implant within the user. The user will be associated with the cochlear implant of a cochlear implant system in the second user state, and thus the user will use the cochlear implant system (instead of the hearing aid device) as a hearing treatment in the second user state.

In some embodiments, to compute the predicted hearing performance of the user in the second user state, the application may apply trained machine learning model 502 to the input dataset of the user, and trained machine learning model 502 may compute one or more predicted hearing performance results of the user with the cochlear implant at one or more timestamps in the second user state (e.g., 1 week after the implant surgical operation, 1 month after the implant surgical operation, etc.) based on the input dataset. In some embodiments, a predicted hearing performance result at a timestamp in the second user state may indicate a hearing performance result that trained machine learning model 502 estimates the user may obtain with the cochlear implant at the timestamp in the second user state. Thus, by applying trained machine learning model 502 on the input dataset of the user who is currently associated with the hearing aid device in the first user state, the application may predict the hearing performance that the user will potentially obtain with the cochlear implant in the second user state after receiving the cochlear implant.

In some embodiments, for each timestamp in the second user state, the application may not only compute the predicted hearing performance result of the user with the cochlear implant at the timestamp, but also compute a confidence range of the hearing performance result of the user at each timestamp. For example, when the application applies trained machine learning model 502 to predict the hearing performance result of the user with the cochlear implant at a timestamp, trained machine learning model 502 may also estimate a value range that likely contains the hearing performance result of the user at the timestamp with a predetermined confidence level (e.g., 95%). In other words, trained machine learning model 502 may determine with the predetermined confidence level that the hearing performance result of the user with the cochlear implant at the timestamp is within the value range, and select such value range to be the confidence range of the hearing performance result of the user with the cochlear implant at the timestamp. In some embodiments, this confidence range computed by trained machine learning model 502 may be relatively small if the number of training examples being used to train machine learning model 502 is relatively large.

In some embodiments, the application may compute the predicted hearing performance of the user in the second user state only if a predetermined condition is satisfied. As an example, the application may determine that the input dataset of the user includes one or more hearing performance results of the user with the hearing aid device that satisfy a predetermined hearing performance threshold (e.g., a speech score of the user is lower than a predetermined score such as 20%), and thus determine that the hearing aid device appears to be relatively ineffective for the user. Accordingly, in response to determining that the input dataset of the user includes one or more hearing performance results that satisfy the predetermined hearing performance threshold, the application may use trained machine learning model 502 to compute the predicted hearing performance that the user may obtain with the cochlear implant.

Alternatively, if the input dataset of the user does not include one or more hearing performance results that satisfy the predetermined hearing performance threshold, the application may determine that the hearing aid device appears to be effective for the user at least to a certain extent. Accordingly, the application may not compute the predicted hearing performance of the user with the cochlear implant in the second user state, and instead display a recommendation to optimize fitting parameters of the hearing aid device or to replace the hearing aid device with another hearing aid device having upgraded operations as described herein.

At operation 806, the application may generate a visual representation of the predicted hearing performance of the user in the second user state. As described herein, the predicted hearing performance of the user may include one or more hearing performance results with the cochlear implant at one or more timestamps that are predicted for the user in the second user state. In some embodiments, the visual representation of the predicted hearing performance may visualize the one or more hearing performance results at the one or more timestamps in the form of a graph, a chart, and/or other graphical representations. In some embodiments, for each timestamp, the visual representation of the predicted hearing performance may also depict the confidence range that likely contains the hearing performance result of the user with the cochlear implant at the timestamp.

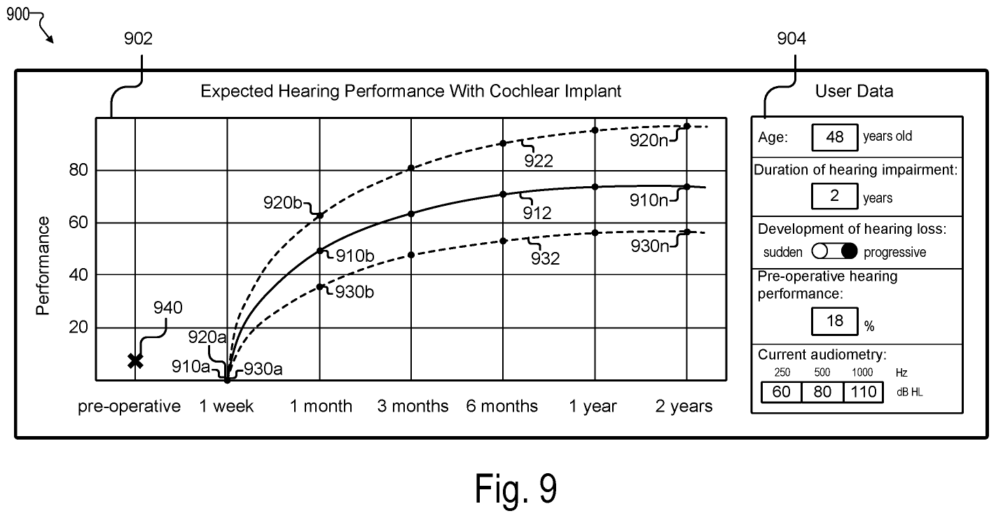

At operation 808, the application may display the visual representation of the predicted hearing performance of the user in the second user state on a display device such as display 306 of computing device 302. FIG. 9 illustrates an example graphical user interface 900 of the application presented on the display device. As depicted in FIG. 9, graphical user interface 900 may include a visual representation 902 of the predicted hearing performance of the user with the cochlear implant in the second user state. Graphical user interface 900 may also include a user data section 904 that comprises one or more graphical elements through which at least some of the input dataset of the user may be provided.

As an example, a hearing specialist may obtain the input dataset from the user, and enter the input dataset of the user into the application via user data section 904. As depicted in FIG. 9, the input dataset of the user may include the user data of the user (e.g., the age of the user, the hearing impairment duration of the user, etc.), the hearing aid dataset of the user (e.g., development of hearing loss, pre-operative hearing performance, current audiometry, etc.), etc. Once the input dataset of the user is provided, the application may apply trained machine learning model 502 to the input dataset to compute the predicted hearing performance of the user with the cochlear implant. Such predicted hearing performance of the user may include various hearing performance results that the user may possibly obtain with the cochlear implant at various timestamps in the second user state as described herein. The application may then generate visual representation 902 of the predicted hearing performance of the user with the cochlear implant, and display visual representation 902 in graphical user interface 900.

As depicted in FIG. 9, visual representation 902 may include one or more first data points 910a . . . 910n (commonly referred to herein as first data points 910) at one or more timestamps that are subsequent to an implant surgical operation to install the cochlear implant within the user (e.g., 1 week after the implant surgical operation, 1 month after the implant surgical operation, 3 months after the implant surgical operation, etc.). In some embodiments, a first data point 910 corresponding to a timestamp may indicate the predicted hearing performance result that the user will potentially obtain with the cochlear implant at the timestamp. As depicted in FIG. 9, first data points 910 may form a predicted performance line 912 on visual representation 902.

As depicted in FIG. 9, visual representation 902 may also include one or more second data points 920a . . . 920n (commonly referred to herein as second data points 920) and one or more third data points 930a . . . 930n (commonly referred to herein as third data points 930) at the one or more timestamps. For each timestamp, second data point 920 at the timestamp may indicate an upper limit value and third data point 930 at the timestamp may indicate a lower limit value of a confidence range that likely contains the hearing performance result of the user with the cochlear implant at the timestamp. As depicted in FIG. 9, second data points 920 may form an upper boundary line 922 on visual representation 902 and third data point 930 may form a lower boundary line 932 on visual representation 902. Thus, upper boundary line 922 and lower boundary line 932 may visually illustrate a value range of the predicted hearing performance results with the cochlear implant at various timestamps that are computed for the user. As depicted in FIG. 9, visual representation 902 may also include a data point 940 indicating the most recent hearing performance of the user. This hearing performance of the user may include a hearing performance result of the user in the most recent hearing performance test that is performed (with or without a hearing aid device) before an implant surgical operation to implant a cochlear implant in the user (if any).

In some embodiments, the user and/or a hearing specialist of the user may reference visual representation 902 to evaluate the predicted hearing performance of the user with the cochlear implant. As described herein, such predicted hearing performance of the user may be objectively estimated using trained machine learning model 502 and visually depicted in visual representation 902. Accordingly, by referencing visual representation 902, the user and/or the hearing specialist may be informed of the hearing performance that the user can potentially obtain with the cochlear implant. Thus, the user can make an informed decision regarding whether to be implanted with the cochlear implant system.

In some embodiments, once a user is implanted within a cochlear implant, an actual hearing performance of the user with the cochlear implant may be monitored.

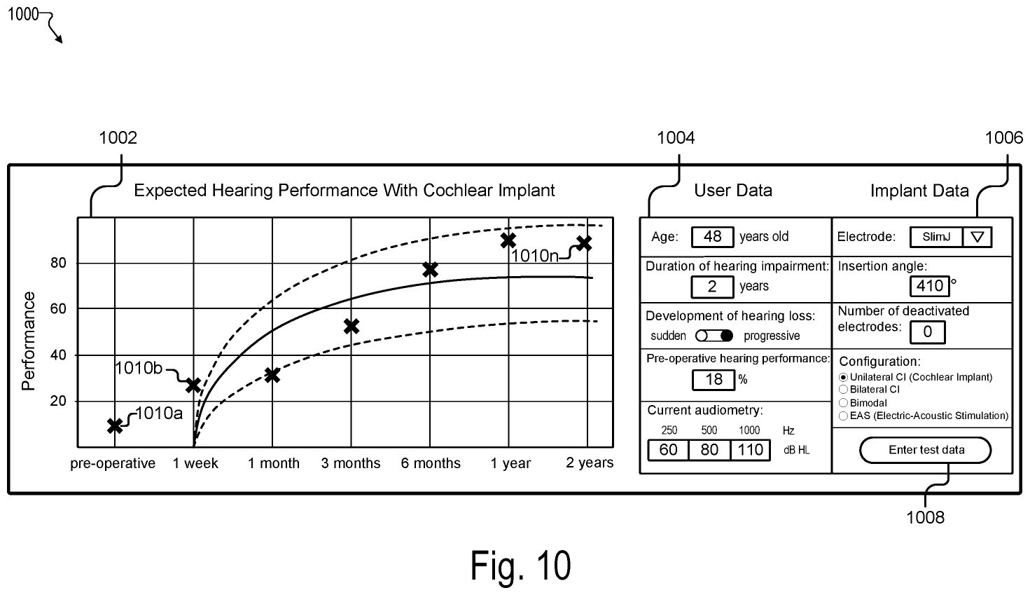

For example, FIG. 10 illustrates an example graphical user interface 1000 that may be generated by an application configured to monitor the hearing performance that the user actually obtains with the cochlear implant. As depicted in FIG. 10, graphical user interface 1000 may include a visual representation 1002, a user data section 1004, and an implant data section 1006. In some embodiments, user data section 1004 may be similar to user data section 904 in FIG. 9 and may include one or more graphical elements through which the input dataset of the user before the implant surgical operation may be entered into the application. In some embodiments, implant data section 1006 may include one or more graphical elements through which implant data of the cochlear implant system implemented for the user may be entered into the application. Non-limiting examples of the implant data include a type of electrode lead implanted within the user, an insertion angle at which the electrode lead is inserted within the user, a number of deactivated electrodes on the electrode lead, a configuration of the cochlear implant system (e.g., unilateral, bilateral, bimodal, etc.), etc.

As depicted in FIG. 10, the implant data section 1006 may include a graphical element 1008 that may be selected to enter a hearing performance of the user with the cochlear implant into the application. Such hearing performance may include one or more hearing performance results of the user in one or more hearing performance tests that are performed with the user using the cochlear implant system at one or more timestamps after the implant surgical operation. In some embodiments, the application may visually illustrate these hearing performance results of the user with the cochlear implant on visual representation 1002. For example, as depicted in FIG. 10, visual representation 1002 may include visual representation 902 and one or more data points 1010a . . . 1010n (commonly referred to herein as data points 1010). Data points 1010 may indicate the hearing performance results of the user with the cochlear implant at one or more timestamps after the cochlear implant is implanted within the user.

As depicted in FIG. 10, data points 1010 may be superimposed on visual representation 902 to form visual representation 1002. As described herein, visual representation 902 may illustrate the predicted hearing performance results of the user with the cochlear implant at the one or more timestamps and also illustrate the confidence range of the hearing performance results that are computed by trained machine learning model 502. Thus, the user and/or the hearing specialist of the user may use visual representation 1002 to evaluate the actual hearing performance results of the user with the cochlear implant relative to the predicted hearing performance results of the user with the cochlear implant. Additionally or alternatively, the user and/or the hearing specialist may use visual representation 1002 to evaluate the actual hearing performance results of the user with the cochlear implant relative to the confidence range predicted to likely contain the hearing performance results of the user with the cochlear implant. Thus, visual representation 1002 may facilitate the evaluation of the actual hearing performance of the user with the cochlear implant after the implant surgical operation, relative to the predicted hearing performance of the user with the cochlear implant computed by trained machine learning model 502 before the implant surgical operation.

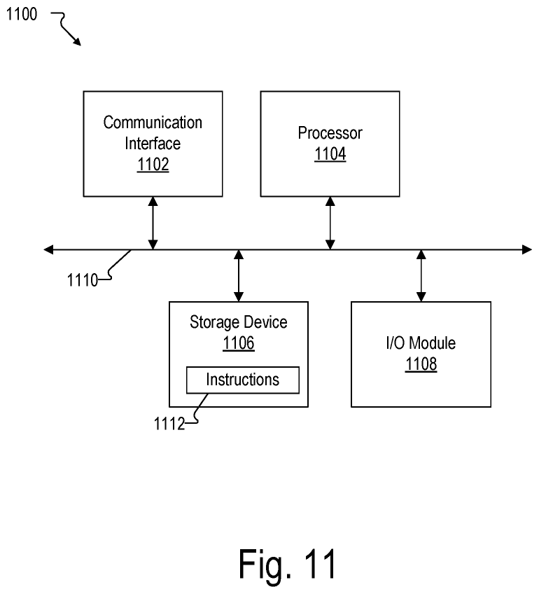

FIG. 11 illustrates an exemplary computing device 1100 that may be specifically configured to perform one or more of the processes described herein. To that end, any of the systems, processing units, and/or devices described herein may be implemented by computing device 1100.

As shown in FIG. 11, computing device 1100 may include a communication interface 1102, a processor 1104, a storage device 1106, and an input/output (“I/O”) module 1108 communicatively connected one to another via a communication infrastructure 1110. While an exemplary computing device 1100 is shown in FIG. 11, the components illustrated in FIG. 11 are not intended to be limiting, Additional or alternative components may be used in other embodiments. Components of computing device 1100 shown in FIG. 11 will now be described in additional detail.

Communication interface 1102 may be configured to communicate with one or more computing devices. Examples of communication interface 1102 include, without limitation, a wired network interface (such as a network interface card), a wireless network interface (such as a wireless network interface card), a modem, an audio/video connection, and any other suitable interface.

Processor 1104 generally represents any type or form of processing unit capable of processing data and/or interpreting, executing, and/or directing execution of one or more of the instructions, processes, and/or operations described herein. Processor 1104 may perform operations by executing computer-executable instructions 1112 (e.g., an application, software, code, and/or other executable data instance) stored in storage device 1106.

Storage device 1106 may include one or more data storage media, devices, or configurations and may employ any type, form, and combination of data storage media and/or device. For example, storage device 1106 may include, but is not limited to, any combination of the non-volatile media and/or volatile media described herein. Electronic data, including data described herein, may be temporarily and/or permanently stored in storage device 1106. For example, data representative of computer-executable instructions 1112 configured to direct processor 1104 to perform any of the operations described herein may be stored within storage device 1106. In some examples, data may be arranged in one or more databases residing within storage device 1106.

I/O module 1108 may include one or more I/O modules configured to receive user input and provide user output. I/O module 1108 may include any hardware, firmware, software, or combination thereof supportive of input and output capabilities. For example, I/O module 1108 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1108 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1108 is configured to provide graphical data to a display for presentation to a user. The graphical data may be representative of one or more graphical user interfaces and/or any other graphical content as may serve a particular implementation.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A method comprising:
aggregating, by a hearing performance prediction system, a plurality of training examples, a training example in the plurality of training examples including a hearing aid dataset and a cochlear implant dataset associated with a user, the hearing aid dataset collected from a clinical facility of the user, a hearing aid device of the user, and an electronic device of the user, the cochlear implant dataset collected from the clinical facility of the user, a cochlear implant of the user, and an electronic device of the user; and
training, by the hearing performance prediction system, a machine learning model using the plurality of training examples by:
computing, using the machine learning model, a predicted hearing performance for the user based on the hearing aid dataset of the user in the training example;
computing a feedback value based on the predicted hearing performance of the user and the cochlear implant dataset of the user in the training example; and
adjusting one or more model parameters of the machine learning model based on the feedback value.

2. The method of claim 1, wherein the aggregating the plurality of training examples includes:
collecting the hearing aid dataset that is generated during a first time period prior to the cochlear implant being implanted in the user, the user being associated with the hearing aid device during the first time period; and
collecting the cochlear implant dataset that is generated during a second time period subsequent to the cochlear implant being implanted in the user, the user being associated with the cochlear implant during the second time period.

3. The method of claim 2, wherein:
the hearing aid dataset includes one or more of: one or more fitting parameters of the hearing aid device, a usage pattern of the user in using the hearing aid device, one or more hearing performance results of the user with the hearing aid device during the first time period, or one or more hearing performance results of the user without the hearing aid device during the first time period.

4. The method of claim 2, wherein the collecting the hearing aid dataset includes one or more of:
receiving, from the clinical facility, one or more fitting parameters of the hearing aid device;
receiving, from one or more of the clinical facility or the electronic device of the user, one or more hearing performance results of the user with the hearing aid device during the first time period and one or more hearing performance results of the user without the hearing aid device during the first time period;
receiving, from the hearing aid device, usage data of the hearing aid device and determining a usage pattern of the user in using the hearing aid device based on the usage data of the hearing aid device.

5. The method of claim 2, wherein:
the cochlear implant dataset includes one or more of: one or more fitting parameters of the cochlear implant, a usage pattern of the user in using the cochlear implant, one or more hearing performance results of the user with the cochlear implant during the second time period, or one or more hearing performance results of the user without the cochlear implant during the second time period.

6. The method of claim 2, wherein the collecting the cochlear implant dataset includes one or more of:
receiving, from the clinical facility, one or more fitting parameters of the cochlear implant;
receiving, from one or more of the clinical facility or the electronic device of the user, one or more hearing performance results of the user with the cochlear implant during the second time period and one or more hearing performance results of the user without the cochlear implant during the second time period; and
receiving, from the cochlear implant, usage data of the cochlear implant and determining a usage pattern of the user in using the cochlear implant based on the usage data of the cochlear implant.

7. The method of claim 1, wherein:
the training example further includes one or more of user data of the user or clinic data of the clinical facility; and
the computing the predicted hearing performance for the user is further based on one or more of the user data of the user or the clinic data of the clinical facility.

8. The method of claim 7, wherein:
the user data of the user includes one or more of: an age of the user, a language of the user, a hearing impairment start point of the user, a hearing impairment duration of the user, a cause of hearing impairment of the user, or one or more test results of one or more tests performed on the user; and
the clinic data of the clinical facility includes a performance metric of the clinical facility.

9. The method of claim 1, further comprising:
determining, by the hearing performance prediction system, that the one or more model parameters of the machine learning model have been sufficiently adjusted; and
implementing, by the hearing performance prediction system in response to the determining that the one or more model parameters of the machine learning model have been sufficiently adjusted, the machine learning model in an application associated with a hearing aid device.

10. A method comprising:
receiving, by an application executed by a computing device, an input dataset of a user in a first user state, wherein the user is associated with a hearing aid device in the first user state;
determining, by the application executed by the computing device, that the input dataset of the user includes a hearing performance result satisfying a hearing performance threshold;
computing, in response to the determining that the input dataset of the user includes the hearing performance result satisfying the hearing performance threshold and by the application executed by the computing device using a trained machine learning model that was trained with one or more hearing aid datasets and one or more cochlear implant datasets, a predicted hearing performance of the user in a second user state based on the input dataset, wherein the user will be associated with a cochlear implant in the second user state;
generating, by the application executed by the computing device, a visual representation of the predicted hearing performance of the user in the second user state; and
presenting, by the application executed by the computing device, the visual representation of the predicted hearing performance on a display device.

11. The method of claim 10, wherein the input dataset of the user in the first state includes one or more of:
a hearing aid dataset of the user including one or more of: one or more fitting parameters of the hearing aid device or a usage pattern of the user in using the hearing aid device; or
user data of the user including one or more of: an age of the user, a language of the user, a hearing impairment start point of the user, a hearing impairment duration of the user, a cause of hearing impairment of the user, or one or more test results of one or more tests performed on the user.

12. (canceled)

13. The method of claim 10, wherein:
the predicted hearing performance of the user includes one or more hearing performance results at one or more timestamps that are predicted for the user in the second user state; and
the visual representation of the predicted hearing performance visualizes the one or more hearing performance results at the one or more timestamps.

14. The method of claim 10, wherein:
the application is executed by the computing device associated with the hearing aid device of the user.

15. The method of claim 10, wherein:
the application is configured to perform one or more of a hearing performance test for the user or a fitting operation for the hearing aid device of the user.

16. A system comprising:
a memory storing instructions;
a processor communicatively coupled to the memory and configured to execute the instructions to:
aggregate, by a hearing performance prediction system, a plurality of training examples, a training example in the plurality of training examples including a hearing aid dataset and a cochlear implant dataset associated with a user, the hearing aid dataset collected from a clinical facility of the user, a hearing aid device of the user, and an electronic device of the user, the cochlear implant dataset collected from the clinical facility of the user, a cochlear implant of the user, and an electronic device of the user; and
train, by the hearing performance prediction system, a machine learning model using the plurality of training examples by:
computing, using the machine learning model, a predicted hearing performance for the user based on the hearing aid dataset of the user in the training example;
computing a feedback value based on the predicted hearing performance of the user and the cochlear implant dataset of the user in the training example; and
adjusting one or more model parameters of the machine learning model based on the feedback value.

17. The system of claim 16, wherein the aggregating the plurality of training examples includes:
collecting the hearing aid dataset that is generated during a first time period prior to the cochlear implant being implanted in the user, the user being associated with the hearing aid device during the first time period; and
collecting the cochlear implant dataset that is generated during a second time period subsequent to the cochlear implant being implanted in the user, the user being associated with the cochlear implant during the second time period.

18. The system of claim 17, wherein:
the hearing aid dataset includes one or more of: one or more fitting parameters of the hearing aid device, a usage pattern of the user in using the hearing aid device, one or more hearing performance results of the user with the hearing aid device during the first time period, or one or more hearing performance results of the user without the hearing aid device during the first time period.

19. The system of claim 17, wherein the collecting the hearing aid dataset includes one or more of:
receiving, from the clinical facility, one or more fitting parameters of the hearing aid device;
receiving, from one or more of the clinical facility or the electronic device of the user, one or more hearing performance results of the user with the hearing aid device during the first time period and one or more hearing performance results of the user without the hearing aid device during the first time period;
receiving, from the hearing aid device, usage data of the hearing aid device and determining a usage pattern of the user in using the hearing aid device based on the usage data of the hearing aid device.

20. The system of claim 17, wherein:
the cochlear implant dataset includes one or more of: one or more fitting parameters of the cochlear implant, a usage pattern of the user in using the cochlear implant, one or more hearing performance results of the user with the cochlear implant during the second time period, or one or more hearing performance results of the user without the cochlear implant during the second time period.

21. The system of claim 17, wherein the collecting the cochlear implant dataset includes one or more of:
receiving, from the clinical facility, one or more fitting parameters of the cochlear implant;
receiving, from one or more of the clinical facility or the electronic device of the user, one or more hearing performance results of the user with the cochlear implant during the second time period and one or more hearing performance results of the user without the cochlear implant during the second time period; and
receiving, from the cochlear implant, usage data of the cochlear implant and determining a usage pattern of the user in using the cochlear implant based on the usage data of the cochlear implant.

22-30. (canceled)
